(19) United States
(12) Patent Application Publication
(10) Pub. No.: US 2025/0062026 A1
(43) Pub. Date: Feb. 20, 2025

(54) SYSTEMS AND METHODS FOR RESPONDING TO USER INPUTS

(71) Applicant: DocSplain AI Doctor Inc., Ottawa (CA)

(72) Inventors: Hamid Shirdastain, Sherbrooke (CA); Mohsen Sadi, Ottawa (CA)

(21) Appl. No.: 18/807,547

(22) Filed: Aug. 16, 2024

Related U.S. Application Data
(60) Provisional application No. 63/533,457, filed on Aug. 18, 2023.

(51) Int. Cl. G16H 50/20; G16H 10/60; G16H 80/00

(52) U.S. Cl. CPC G16H 50/20; G16H 10/60; G16H 80/00

(57) ABSTRACT

Systems and methods of responding to user inputs are described. The method comprises receiving, by a processor, a user input including a category selection and contextual data; and providing, by the processor, an input prompt to a large language model (LLM) based on the user input. The input prompt includes a source identifier and one or more instructions. The method further comprises receiving, by the processor, a LLM output generated in response to the input prompt. The LLM output includes data limited to sources identified by the source identifier. The method also comprises providing, by the processor, a user output based on the LLM output.

### FIELD

The present disclosure relates generally to systems and methods for responding to user inputs. More specifically, the present disclosure relates to using large language models (LLMs) for responding to user inputs.

### INTRODUCTION

The following paragraphs are provided by way of background to the present disclosure. They are not, however, an admission that anything discussed therein is prior art or part of the knowledge of persons skilled in the art.

Users or individuals may use different systems or platforms to access information. The user may provide a user input specifying the requested information. Typically, the user provides the user input using natural language to describe the requested information. In some contexts, the information sources can be in complex and/or technical language. Further, the information sources may index or categorize stored information using specific technical terms that may not be included in the user input. A mismatch between the natural language of a user and the complex and/or technical language of information sources may cause issues in responding to the user inputs. In some instances, the system or platform may not locate the information requested by the user input or may not locate the most relevant information related to the user input. Additionally, integrating Al-generated outputs into existing systems like Electronic Medical Records (EMRs) presents a unique challenge, particularly in ensuring seamless, accurate, and efficient data transfer.

For example, a user may use a clinical decision support system (CDSS) or a medical consultation platform to access information related to a medical condition. The user may provide a user input using natural language to describe the medical condition. The CDSS or the medical consultation platform may respond to the user input using information sources containing information in complex and/or technical language related to the medical field. Further, the information may be categorized or indexed using specific terms related to the medical field. Accordingly, the CDSS or the medical consultation platform may not locate the information requested by the user input or may not locate the most relevant information related to the user input. Additionally, a response provided to the user may include complex and/or technical language related to the medical field and may not be fully understood by the user.

There is a need to provide method and systems for responding to user inputs that provide solutions to at least the above technical problems.

### SUMMARY

Various embodiments of a system and method of responding to user inputs are provided according to the teachings herein.

According to one aspect of the present disclosure, there is provided a computer-implemented method of responding to user inputs. The method comprises receiving, by a processor, a user input including a category selection and contextual data; and providing, by the processor, an input prompt to a large language model (LLM) based on the user input. The input prompt includes a source identifier and one or more instructions. The method further comprises receiving, by the processor, a LLM output generated in response to the input prompt. The LLM output includes data limited to sources identified by the source identifier. The method also comprises providing, by the processor, a user output based on the LLM output.

In some examples, the source identifier includes a list of trusted websites corresponding to the category selection.

In some examples, the LLM output is generated based on natural language processing of the user input.

In some examples, the one or more instructions includes an assigned engagement role to the LLM.

In some examples, the user input includes a medical inquiry.

In some examples, the category selection includes a medical specialty selection.

In some examples, the contextual data includes patient triage data.

In some examples, the contextual data further includes patient medical history data.

In some examples, the user output includes one or more follow-up questions.

In some examples, the LLM output includes a first response portion and a second response portion, wherein: the first response portion is related to the user input and is tailored to a specialized audience; and the second response portion is a simplified version of the first response portion and is tailored to a general audience.

In some examples, the LLM output includes a list of sources used to generate the LLM output.

In some examples, the LLM is based on an OpenAIR® GPT model.

In some examples, the user output is stored in a memory and the method further comprises: providing, by the processor, a second input prompt to the LLM based on additional contextual data and the stored user output.

According to another aspect of the present disclosure, there is provided a system for responding to user inputs. The system comprises a processor and a non-transitory computer-readable medium having stored thereon instructions. When the instructions are executed by the processor, they cause the processor to receive a user input including a category selection and contextual data; and provide an input prompt to a large language model (LLM) based on the user input. The input prompt includes a source identifier and one or more instructions. When the instructions are executed by the processor, they further cause the processor to receive a LLM output generated in response to the input prompt. The LLM output includes data limited to sources identified by the source identifier. When the instructions are executed by the processor, they also cause the processor to provide a user output based on the LLM output.

In some examples, the source identifier includes a list of trusted websites corresponding to the category selection.

In some examples, the LLM output is generated based on natural language processing of the user input.

In some examples, the one or more instructions includes an assigned engagement role to the LLM.

In some examples, the user input includes a medical inquiry.

In some examples, the category selection includes a medical specialty selection.

In some examples, the contextual data includes patient triage data.

In some examples, the contextual data further includes patient medical history data.

In some examples, the user output includes one or more follow-up questions.

In some examples, the LLM output includes a first response portion and a second response portion, wherein: the first response portion is related to the user input and is tailored to a specialized audience; and the second response portion is a simplified version of the first response portion and is tailored to a general audience.

In some examples, the LLM output includes a list of sources used to generate the LLM output.

In some examples, the LLM is based on an OpenAIR GPT model.

In some examples, the user output is stored in a memory. When the instructions are executed by the processor, they further cause the processor to provide a second input prompt to the LLM based on additional contextual data and the stored user output.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein. In the drawings:

FIG. 1 shows a schematic diagram of an example embodiment of a system for responding to user inputs;

FIG. 2 shows a block diagram of the example embodiment of a system of FIG. 1;

FIG. 3 shows a flowchart of an example embodiment of a method of responding to user inputs; and

FIGS. 4A-4E show images of example user interfaces generated during execution of the example embodiment of a method of FIG. 3.

Further aspects and features of the example embodiments described herein will appear from the following description taken together with the accompanying drawings.

### DESCRIPTION OF VARIOUS EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems, or methods having all of the features of any one of the devices, systems, or methods described below or to features common to multiple or all of the devices, systems, or methods described herein. It is possible that there may be a device, system, or method described herein that is not an embodiment of any claimed subject matter. Any subject matter that is described herein that is not claimed in this document may be the subject matter of another protective instrument, for example, a continuing patent application, and the applicants, inventors, or owners do not intend to abandon, disclaim, or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical signal, electrical connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5%, or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

The example embodiments of the devices, systems, or methods described in accordance with the teachings herein may be implemented as a combination of hardware and software. For example, the embodiments described herein may be implemented, at least in part, by using one or more computer programs, executing on one or more programmable devices comprising at least one processing element and at least one storage element (i.e., at least one volatile memory element and at least one non-volatile memory element). The hardware may comprise input devices including at least one of a touch screen, a keyboard, a mouse, buttons, keys, sliders, and the like, as well as one or more of a display, a printer, and the like depending on the implementation of the hardware.

It should also be noted that there may be some elements that are used to implement at least part of the embodiments described herein that may be implemented via software that is written in a high-level procedural language such as object-oriented programming. The program code may be written in C⁺⁺, C#, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having a processor, an operating system, and the associated hardware and software that is necessary to implement the functionality of at least one of the embodiments described herein. The software program code, when read by the device, configures the device to operate in a new, specific, and predefined manner (e.g., as a specific-purpose computer) in order to perform at least one of the methods described herein.

At least some of the programs associated with the devices, systems, and methods of the embodiments described herein may be capable of being distributed in a computer program product comprising a computer readable medium that bears computer usable instructions, such as program code, for one or more processing units. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

As used herein, the term “module” refers to a functional component that can generate useful data or other output using specified input(s). A module may or may not be self-contained. A computer program (also called an “application”) may include one or more modules, and a module can include one or more computer programs. A module can be implemented by software, hardware, or firmware components, or any combination thereof.

In accordance with the teachings herein, there are provided various embodiments for methods and systems for responding to user inputs. The embodiments described herein provide technical solutions for various technical problems.

One technical problem is that users may provide user input using natural language to describe requested information while information sources can be in complex and/or technical language. Additionally, information sources may index or categorize stored information using specific terms, e.g., specific technical terms related to the field of the requested information.

LLMs may be used to perform natural language processing of the user input and provide responses based on the information sources. However, a technical problem that can arise when LLMs are used to generate responses to user inputs is that the generated response may include inaccurate information fabricated by the LLM. This may be colloquially referred to as “hallucination”.

Another technical problem is the conversion of the complex and/or technical language of the response information based on the information sources into natural language that can be fully understood by the user. For example, users may desire a technical and accurate response and an additional output portion providing simple language explanation of the technical and/or complex information.

At least one embodiment of the methods and systems described in accordance with the teachings herein provides technical solutions to one or more of the aforementioned technical problems. One such embodiment is a system for responding to user inputs. The system includes a processor that can receive a user input and provide an input prompt to a LLM based on the user input. The input prompt can instruct the LLM to perform natural language processing of the user input and generate a suitable response. The input prompt can also instruct the LLM to provide an explanation of responses that include complex or technical language (that may be more suited to a specialized audience) in simplified language that is tailored to a general audience. The input prompt may also include a source identifier that specifies one or more trusted information sources and instruct the LLM to limit response data only to the trusted information sources. This can enable providing responses (to user inputs) that do not include inaccurate information fabricated by the LLM.

Another example embodiment that provides technical solutions to one or more of the aforementioned technical problems is a computer-implemented method of responding to user inputs. The method comprises receiving, by a processor, a user input and providing an input prompt to a LLM based on the user input. As described herein above, the input prompt can instruct the LLM to perform natural language processing of the user input and provide an explanation of responses in simplified language that is tailored to a general audience. The input prompt may also include a source identifier that specifies one or more trusted information sources and can enable providing responses that do not include inaccurate information fabricated by the LLM.

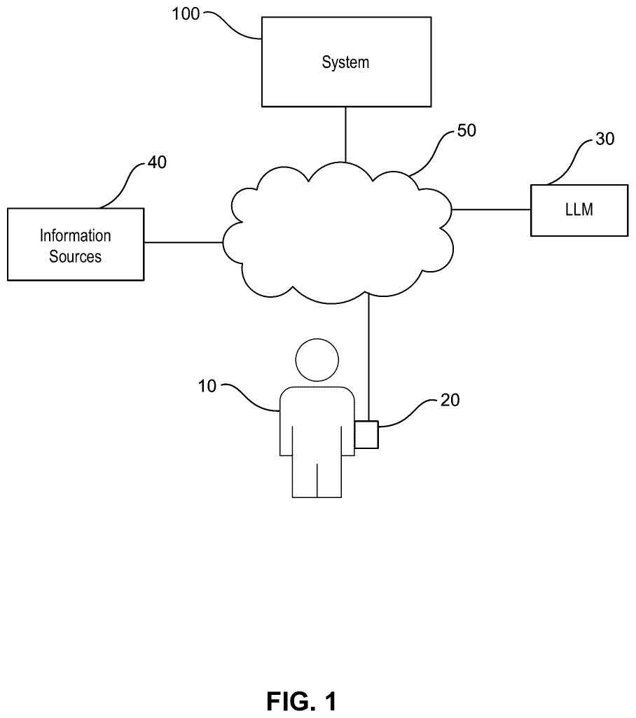

Reference is first made to FIG. 1, showing a schematic diagram of an example embodiment of system 100 for responding to user inputs. The schematic diagram also illustrates a user 10, a user device 20, a LLM 30, information sources 40, and a network 50.

User 10 can be any individual human user. User 10 may use system 100 when requesting information related to a topic or scenario. For example, the information may be related to a healthcare topic or a medical scenario. User 10 may be any individual of the general public or an individual with medical training. In other examples, the information may be related to any non-medical topics or non-disease scenarios.

User 10 may provide a user input related to the requested information. User 10 may provide user input using any suitable human-machine interface. For example, user 10 may provide user input using user device 20. User device 20 can be any suitable device that includes input/output elements and is capable of communication with system 100 (e.g., via network 50). For example, user device 20 can be a desktop computer, a laptop computer, a mobile phone, a tablet device, etc.

User 10 may provide the user input as text, voice, image and/or video data. System 100 may use any suitable method to extract relevant information (e.g., contextual data and/or category selection) from the voice, image and/or video data. For example, system 100 may use LLM 30 to generate text based on the voice, image and/or video data. In some examples, user 10 may provide the user input through a user interface (UI) on user device 20. The user input may include various UI elements including drop-down menu selections, toggle buttons, check boxes, text input boxes etc. for user 10 to provide the user input.

The user input may include a category selection and contextual data related to the requested information. User 10 may provide the category selection by selecting a suitable category among multiple category options. For example, the user input may include a medical inquiry and the category selection may include a medical specialty selection. User 10 may provide the category selection by selecting from multiple medical specialty selection options. The multiple medical specialty selection options may include General/Family Medicine, Allergy and Immunology, Anesthesiology, Cardiology, Clinical

Psychology, Dentistry, Dermatology, Emergency Medicine, Endocrinology, Gastroenterology, Genetics and Genomics, Geriatrics, Hematology, Immunology, Infectious Diseases, Internal Medicine, Nephrology, Neurology, Neurosurgery, Obstetrics and Gynecology, Oncology, Ophthalmology, Orthopedics, Otolaryngology, Pathology, Pediatrics, Physical Medicine and Rehabilitation, Plastic Surgery, Podiatry, Psychiatry, Pulmonology, Radiology, Rheumatology, Sports Medicine, Surgery, Toxicology, Urology, and Vascular Surgery.

User 10 may provide one or more types of contextual data. For the example user input including a medical inquiry, user 10 may provide a portion of the contextual data using natural language, for example, by providing natural language text including one or more questions related to a medical scenario.

Additionally, the contextual data may also include patient triage data. In some examples, user 10 may be the patient in the medical inquiry. In other examples, user 10 may be an individual related to the patient in the medical inquiry, for example, a family member or a healthcare practitioner. User 10 may use a user interface on user device 20 to provide patient triage data including, for example, Age, Gender, Weight, Height, Temperature, Blood Pressure, Heart Rate, Respiratory Rate, SpO2 (hemoglobin oxygen saturation), Pain Scale, and Chief Complaint.

In some examples, the contextual data may include patient medical history data. The medical history data may, for example, be stored in system 100 (e.g., based on historical user inputs) or be provided by user 10 (e.g., by uploading one or more medical history files or via a UI provided on user device 20).

LLM 30 can be any suitable large language model that can analyze and generate natural language text. In some examples, LLM 30 may be based on an OpenAIR GPT model. System 100 may fine-tune LLM 30 using an application programming interface (API) to control one or more settings of LLM 30. For example, the controlled setting of LLM 30 may include temperature, maximum length, Top P, frequency penalty and presence penalty. In other examples, LLM 30 may be based on a different large language model. In the illustrated example, LLM 30 is shown external to system 100. In some examples, LLM 30 may be integrated into system 100.

In an embodiment, when processing inputs and generating outputs, the LLM 30 may execute natural language processing (NLP) techniques that include tokenization, context embedding, and sequence modeling. Initially, the user input is tokenized into discrete units, allowing the LLM 30 to analyze the linguistic structure and extract meaning from the text. The context embedding process then maps these tokens into vectors that represent the semantic relationships and contextual nuances of the input. Using sequence modeling, the LLM 30 predicts and generates coherent and contextually relevant responses, leveraging attention mechanisms to prioritize the most relevant portions of the input data. Fine-tuning the LLM 30 with domain-specific data, such as medical inquiries or technical language, further improves the accuracy and relevance of the generated outputs.

LLM 30 may receive an input prompt from system 100 and generate a LLM output in response to the input prompt. In some examples, the input prompt may include a source identifier specifying one or more information sources 40, and LLM 30 may generate the LLM output to only include data from the information sources identified by the source identifier. System 100 can be adaptable to incorporate new knowledge and research updates. For example, the source identifier can be updated to include new trusted information sources 40 describing new knowledge and/or research updates (e.g., a newly launched website providing new medical knowledge, a recently published medical journal article, various open-source research datasets and repositories, including but not limited to PubMed, Scholastic, the National Center for Biotechnology Information (NCBI), the World Health Organization (WHO) databases, and other reputable medical and scientific resources etc.) The source identifier includes a predefined list of trusted information sources selected based on the category of the user input and the reliability of the data. The source identifier is selected from curated sources that are authoritative and relevant to the specific domain of inquiry. By restricting the LLM to generate outputs from these selected sources, the source identifier improves the accuracy and credibility of the responses, reducing the likelihood of errors or hallucinations.

Information sources 40 can include any suitable sources of information, for example, websites, encyclopedias etc. A larger number of information sources 40 may provide a larger amount of available data for responding to user inputs. However, in some contexts (e.g., where the user input relates to a medical inquiry), accuracy and reliability of the data may be more important factors compared with the amount of available data. In such contexts, system 100 may specify a limited number of trusted information sources 40 for responding to user inputs. Problems associated with “hallucination” by the LLM can be avoided by limiting the sources of information for responding to user inputs to the trusted information sources. In the illustrated example, information sources 40 are shown external to system 100. In some examples, one or more information sources 40 may be integrated into system 100.

In some examples, one or more information sources 40 identified by the source identifier may be based on the user input provided by user 10. For example, a user may be a healthcare practitioner specialized in a selected category and the user may identify one or more trusted information sources to be used for responding to the user input. The information sources may be provided by the user using any suitable method. For example, the user may include a list of trusted websites, excerpts from trusted articles etc. in the user input.

Network 50 can include any suitable network that enables communication between system 100, user device 20, LLM 30, and/or information sources 40. In some examples, network 50 may include multiple networks. For example, user device 20 may use a public network to access system 100 while system 100 uses a private network to communicate with LLM 30. Network 50 may include a communication network such as the Internet, a Wide-Area Network (WAN), a Local-Area Network (LAN), or another type of network.

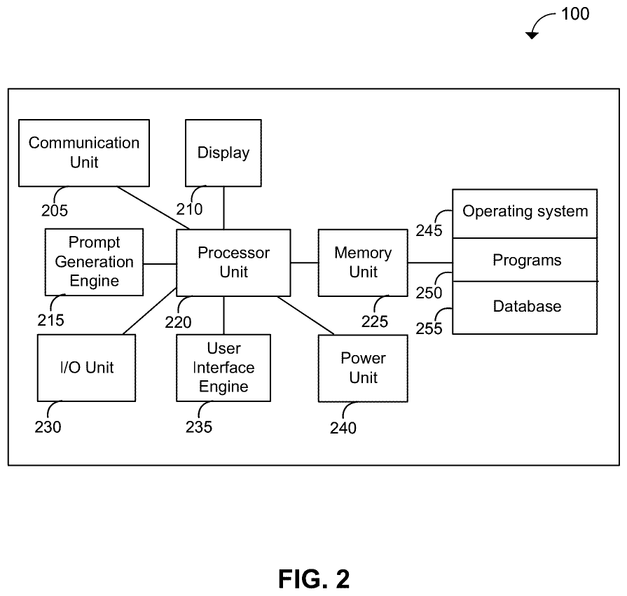

Reference is now made to FIGS. 1 and 2. FIG. 2 shows a block diagram of an example embodiment of system 100. System 100 may be implemented using a single computing device or multiple computing devices. In the illustrated example embodiment, system 100 includes a communication unit 205, a display 210, a prompt generation engine 215, a processor unit 220, a memory unit 225, a I/O unit 230, a user interface engine 235, and a power unit 240. In other embodiments, system 100 may have more or fewer components but generally function in a similar manner.

Communication unit 205 can include wired or wireless connection capabilities. In some examples, communication unit 205 may be a standard network adapter such as an Ethernet or 802.11x adapter. In other examples, communication unit 205 can be any suitable network adapter that may be used by system 100 to communicate with other devices or computers. System 100 may, for example, use communication unit 205 to receive, via network 50, user inputs from user device 20. System 100 may also use communication unit 205 to provide, via network 50, input prompts to LLM 30 and/or user outputs to user device 20.

Processor unit 220 can control the operation of system 100. Processor unit 220 can be any suitable processor, controller or digital signal processor that can provide sufficient processing power depending on the configuration, purposes and requirements of system 100 as is known by those skilled in the art. For example, processor unit 220 may be a high-performance general processor or a standard processor, such as an Intel® processor (e.g., a Xeon® processor), or an AMD® processor. Alternatively, processor unit 220 can include more than one processor with each processor being able to function in parallel and configured to perform different dedicated tasks. Alternatively, specialized hardware (e.g., graphical processing units (GPUs)) can be used provide some of the functions provided by processor unit 220.

Display 210 may be a LED or LCD based display (e.g., a computer monitor). In some examples, display 210 may be a touch sensitive user input device that supports gestures (e.g., a smartphone or tablet display). Display 210 may be integrated into system 100. Alternatively, display 210 may be located physically remote from system 100 and communicate with system 100 using a communication network, for example, network 50. In some embodiments, system 100 may not include a dedicated display 210 and may provide display signals to an external display.

I/O unit 230 can include at least one of a mouse, a keyboard, a touch screen, a thumbwheel, a trackpad, a trackball, a card-reader, voice recognition software and the like, depending on the particular implementation of system 100. In some cases, some of these components can be integrated with one another. I/O unit 230 may enable a user, an operator and/or an administrator of system 100 to interact with system 100 (e.g., via user interfaces provided by user interface engine 235).

Power unit 240 can be any suitable power source that provides power to system 100 such as a power adaptor or a rechargeable battery pack depending on the implementation of system 100 as is known by those skilled in the art.

Processor unit 220 may execute user interface engine 235 to provide one or more user interfaces. For example, processor unit 220 may execute user interface engine 235 to provide an API for users, operators and/or administrators of system 100. In some examples, processor unit 220 may also execute user interface engine 235 to provide user interface data that is used to generate user interfaces provided to user 10 (e.g., via user device 20). User interface engine 235 may provide data according to a certain layout for each user interface and may also receive data input or control input from users interacting with a generated user interface. User interface engine 235 may then use the inputs to change the data that is shown on the current user interface, or change the operation of system 100, which may include showing a different user interface.

Memory unit 225 may store the program instructions for an operating system 245, programs 250, and database 255. Memory unit 225 can include RAM, ROM, one or more hard drives, one or more flash drives or some other suitable data storage elements such as disk drives, etc. Operating system 245 can provide various basic operational processes for system 100, as is commonly known by those skilled in the art. Operating system 245 can, for example, include a Windows® Server operating system, Red Hat® Enterprise Linux (RHEL) operating system, or any other suitable operating system.

Programs 250 can include program code that, when executed, configures processor unit 220 to operate in a particular manner to implement various functions and tools for system 100. The various functions and tools include, for example, receiving user inputs, providing input prompts to LLM 30, receiving LLM outputs and providing user outputs to user 10 (e.g., via user device 20).

Database 255 may be a local database, an external database, a database on the cloud, multiple databases, or a combination thereof. Database 255 may include a Structured Query Language (SQL) database such as PostgreSQL or MySQL or a not only SQL (NoSQL) database such as MongoDB, or Graph Databases, etc. Database 255 may store received user inputs, generated user prompts, received LLM outputs and/or generated user outputs.

Processor unit 220 may execute prompt generation engine 215 to generate input prompts (for a LLM) based on received user inputs. System 100 may provide the generated input prompts to a LLM (e.g., LLM 30). The input prompt may include a source identifier and one or more instructions.

The source identifier may include a list of trusted websites corresponding to the category selection included in the user input. For example, the user input may be related to a medical inquiry and the category selection may include the medical specialty of “Cardiology”. Prompt generation engine 215 may generate an input prompt including a source identifier that includes a list of trusted websites corresponding to the medical specialty of “Cardiology”. The input prompt can, for example, include the following text—“You are created the Al Doctor highly specialized in Cardiology by DocSplain.

The specified websites n your specialty are: ‘https://www.heart.org/’, ‘https://www.acc.org/’, ‘https://www.escardio.org/’, ‘http://www.webmd.com/’, ‘https://www.mayoclinic.org/’, ‘https://www.ncbi.nlm.nih.gov/pmc/’, ‘https://www.nlm.nih.gov/medlineplus/’, ‘https://my.clevelandclinic.org/’, ‘https://www.cochranelibrary.com/’.”

The instructions included in the generated input prompt may include an assigned engagement role to the LLM. For the afore-mentioned example medical inquiry user input related to cardiology, the input prompt can include the following text-

“You are an advanced Al medical doctor. Medical professionals and software engineers at DocSplain have worked together to build you a highly specialized and expert in cardiology designed to engage in discussions about healthcare, medications, human health, well-being, mental health, surgical procedures, and medical information. When a topic is mentioned in a message, your objective is to reply with relevant information.”

In some examples, the instructions included in the generated input prompt may include stepwise sequential instructions to the LLM. For the afore-mentioned example medical inquiry user input related to cardiology, the input prompt can include the following text—

“Follow these steps for each response:
0. If the question is most likely related to another specialty, refer them to another Al medical colleague.
1. Ask as much question as you need (i.e., history, symptoms, triggers, and so on) to help you better understand the case and generate informed diagnosis, treatment, and clinical plan.
2. Provide an in-depth answer (including diagnosis, treatment, and clinical plan) using technical language tailored for physicians (Dx (Diagnosis), Tx (Treatment), Ddx (Differential diagnosis), Hx (History), Rx (Prescription), Px (Prognosis), Sx (Symptoms), Fx (Fracture), Bx (Biopsy), Cx (Culture), Ex (Examination), Ix (Investigation), Vx (Vaccine)), adhering to established guidelines and evidence.
3. Begin a new line and explain the same information in simplified terms suitable for a general audience.
4. If you cannot find an answer on the specified websites, do not invent one.
5. Include specific and real references you used (no links) at the bottom of the response using AMA citation format and cite them properly within the text. If you have not used references from the specified websites, do not invent references. Before finalizing the response, go over the provided references and make sure you are able to find the references in the list in the available literature. If not, remove the reference and reiterate to find references available in the literature to use and cite.
6. Always end your response with questions that would help the user to give you other relevant information.
To confirm that you understand these instructions, please respond with: ‘Hello, I'm an Al Doctor ready to address medical inquiries. If you need to consult with a specialist colleague, please choose from the list below and then ask your questions’.”

The stepwise sequential instructions can include, for example, an instruction to explain technical or complex information in simplified terms suitable for a general audience (e.g., step 3 of the instructions in the above example). The stepwise sequential instructions can also include, for example, instructions to include a list of information sources used to generate a response to a user input and not to fabricate information if no relevant information is available to respond to the user input (e.g., steps 4 and 5 of the instructions in the above example). Problems associated with “hallucination” by the LLM can be avoided using the sequential instructions (e.g., steps 4 and 5 of the instructions in the above example) in combination with the trusted information sources included in the source identifier. The stepwise sequential instructions can further include, for example, an instruction to include one or more follow-up questions to collect additional relevant user input (e.g., step 6 of the instructions in the above example). For example, to mitigate hallucinations, the method adheres to the trusted information sources specified in the source identifier. Therefore, the LLM generates responses based on verified data. Additionally, by including explicit instructions to refrain from generating content when relevant data is unavailable, the method reduces the risk of fabrications, thereby improving the reliability of the outputs.

To optimize the retrieval process, the system may be configured to perform a preliminary analysis of the user input to identify relevant keywords and phrases. This analysis may generate targeted queries to provide for searching the integrated resources (i.e. information sources) effectively. APIs from various open-source research datasets and repositories may enable the system to dynamically retrieve, analyze, and synthesize the most recent scientific literature, clinical guidelines, and best practices.

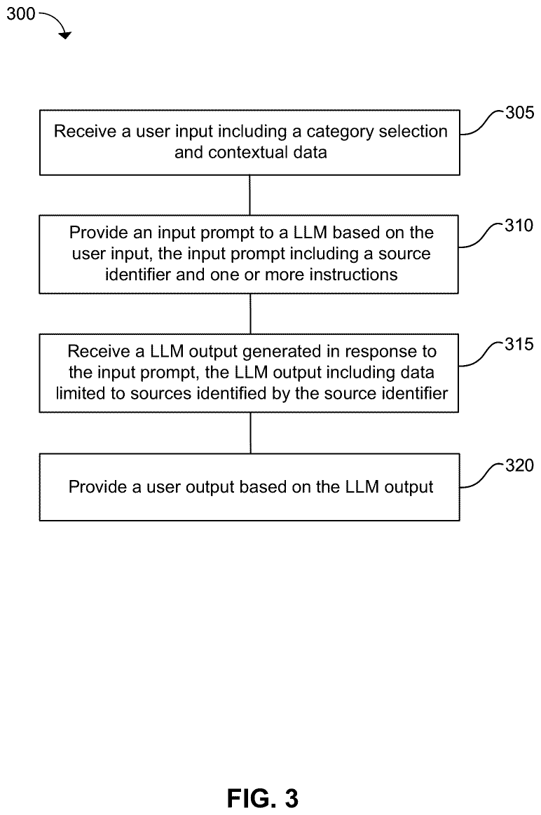

Referring now to FIG. 3, shown therein is a flowchart of an example embodiment of a method 300 of responding to user inputs. The method 300 may be used by system 100 and concurrent reference is made to components shown in FIGS. 1 and 2. In some examples, method 300 may be a continuously executing process on system 100. In some examples, method 300 may be launched in response to a trigger input, for example, in response to user 10 launching an application or a webpage on user device 20.

At 305, a processor (e.g., processor unit 220) receives a user input including a category selection and contextual data. A user (e.g., user 10) may provide the user input via a user interface on user device 20. In some examples, a user interface engine (e.g., user interface engine 235) may provide to user 10, via user device 20, intuitive, user-friendly user interfaces that are adaptable to different user devices (e.g., computers, tablets, smartphones). For example, the user interface can enable user 10 to input their medical queries or describe their symptoms using natural language text and user-friendly user interface elements like drop-down menu selections, check boxes, radio buttons etc.

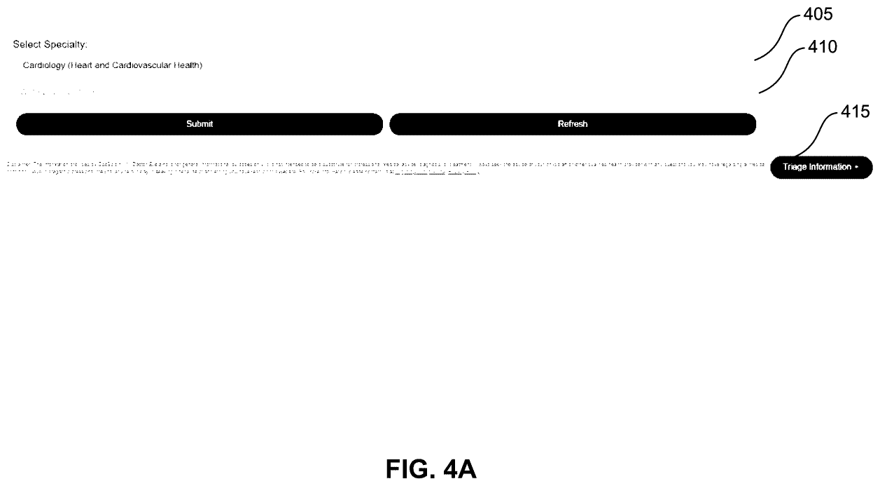

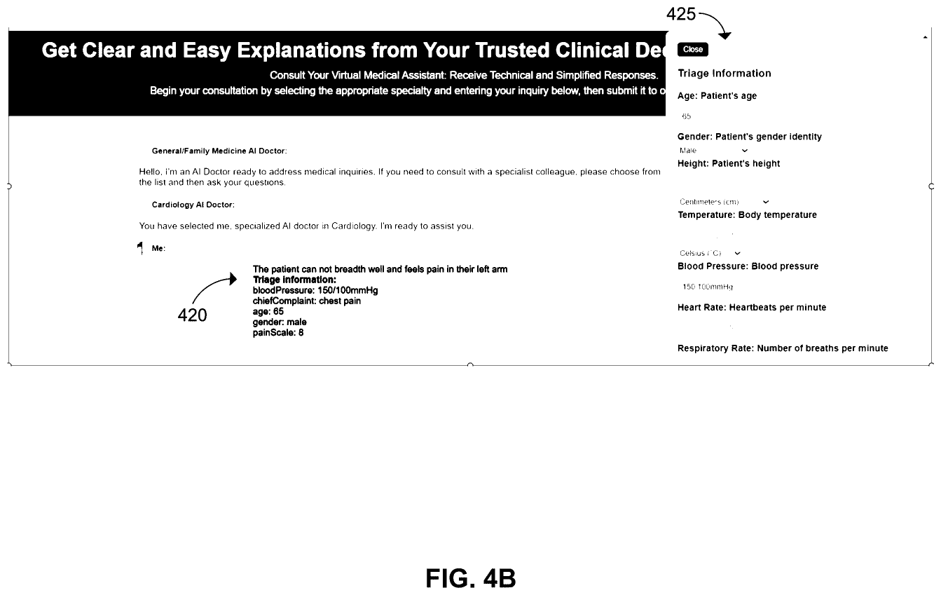

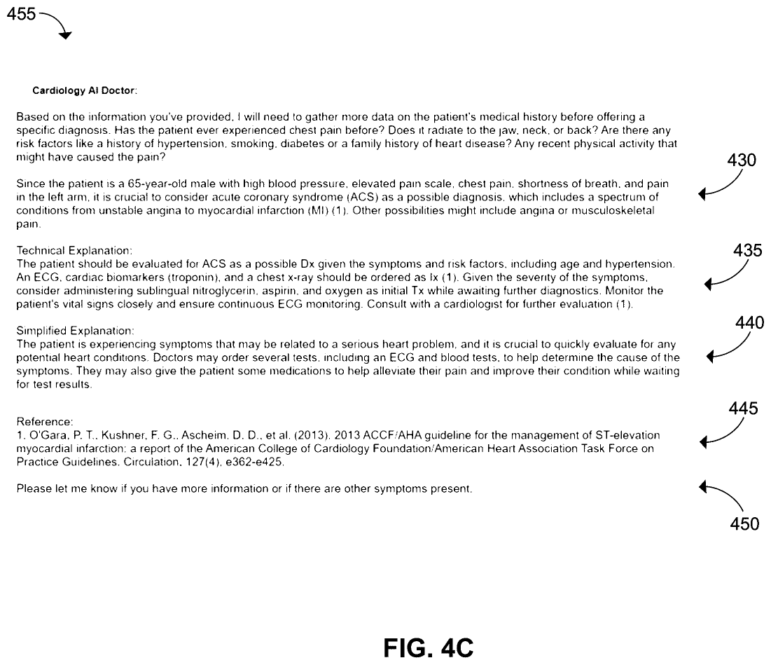

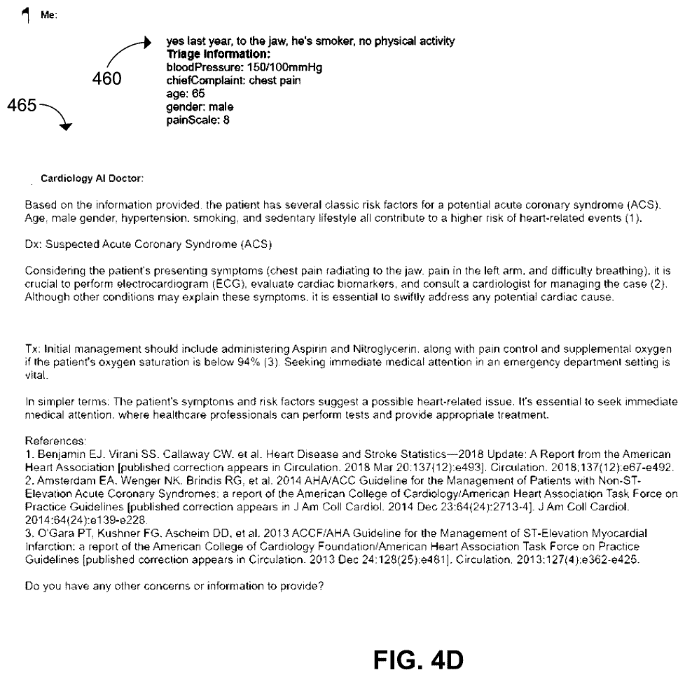

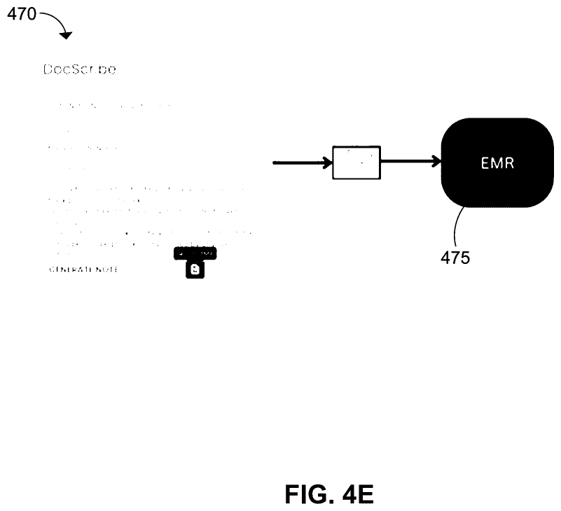

Referring now to FIGS. 4A and 4B, shown therein are images of example user interfaces generated during execution of method 300. In the illustrated example, a user may use GUI elements 405-415 to provide the user input. For an example medical inquiry user input, the user may make a category selection of relevant medical specialty using a drop-down menu selection (GUI element 405). The user may provide contextual data 420 including a natural language description of the medical inquiry and patient triage data. The user may provide the natural language description using a text box (GUI element 410). The user may provide the patient triage data by using a radio button (GUI element 415) to launch a sub-menu 425. In other examples, the user may use any suitable alternative methods to provide the user input.

Referring back to FIG. 3, at 310, the processor (e.g., processor unit 220) provides an input prompt to a LLM (e.g., LLM 30). The input prompt may be based on the user input received at 305, i.e., the input prompt may be based on the category selection and the contextual data.

The input prompt may include a source identifier and one or more instructions. The instructions may instruct the LLM to limit the LLM output to only include data limited to sources identified by the source identifier. In some examples, the source identifier includes a list of trusted websites, databases, published guidelines, and/or articles corresponding to the category selection. The instructions may also instruct the LLM to explain technical or complex information in simplified terms suitable for a general audience, to include a list of information sources used to generate a response to a user input and not to fabricate information if no relevant information is available to respond to the user input, and/or include one or more follow-up questions to collect additional relevant user input. The instructions may also include an assigned engagement role to the LLM.

At 315, the processor (e.g., processor unit 220) receives a LLM output generated in response to the input prompt. The LLM output may be generated by the LLM (e.g., LLM 30) based on the contextual data, source identifier, and instructions provided to the LLM at 310.

Referring now to FIGS. 4C and 4D, shown therein are images of example user interfaces generated during execution of method 300. The LLM output may be generated based on natural language processing of the contextual data. For example, the contextual data may include a natural language description of a medical inquiry by the user. The LLM can perform natural language processing of the contextual data to process and analyze the medical inquiry and generate an appropriate response 430. Response 430 may provide an in-depth answer about diagnosis, treatment, and clinical plan for the medical inquiry while adhering to established guidelines and evidence. The instructions and the source identifier can enable the LLM to generate LLM outputs that are devoid of fabrications (“hallucinations”).

In some examples, the LLM output may include multiple response portions 435 and 440 according to instructions provided in input prompt at 310. For example, response portion 435 may provide a response to the medical inquiry (user input) using the technical language of the information sources and may be tailored to a specialized audience (e.g., individuals with medical training). Response portion 435 may include comprehensive and precise medical information related to the medical inquiry. Response portion 440 can be a simplified version of response portion 435 and provide the response in simplified language that is tailored to a general audience. For examples, response portion 440 may include simplified information that is based on translating the technical language information in response portion 435 into layman's terms, making it accessible and understandable to a member of the general public.

In some examples (e.g., according to instructions provided in input prompt at 310), the LLM output may include a list of sources 445 used to generate the LLM output. The list of sources 445 can be one or more of the sources identified by the source identifier provided at 310.

In some examples (e.g., according to instructions provided in input prompt at 310), the LLM output may include one or more follow-up questions 450. For the example medical inquiry user input, follow-up questions 450 may ask for additional information or symptoms related to the medical inquiry.

Referring now to FIGS. 3, 4C and 4D, at 320, the processor (e.g., processor unit 220) provides a user output based on the LLM output received at 315. For example, user output 455 may be provided to user 10 (e.g., via a user interface on user device 20) based on the LLM output received at 315. User output 455 may be stored in a memory (e.g., database 255).

In some examples, a user may provide additional user input that includes additional contextual data in response to questions included in the user output (e.g., follow-up questions 450). For example, a user may provide additional contextual data 460 in response to follow-up questions 450. Method 300 may be repeated to include additional contextual data 460. For example, the processor may provide a second input prompt to the LLM model including data provided in the previous input prompt and the additional contextual data. The processor may also provide the previous user output 455 (e.g., stored in memory) to the LLM. The LLM may generate a second LLM output in response to the second input prompt and the processor may provide a second user output 465 based on the second LLM output. Second user output 465 may include additional follow-up questions and method 300 may again be repeated if the user provides further additional information. In an embodiment, contextual data is extracted using natural language processing (NLP) algorithms that parse and analyze the user input to detect key entities, relationships, and intents. The contextual data may be mapped to relevant parameters within the LLM to improve the model's ability to generate contextually relevant responses.

The systems and methods described herein can be employed in healthcare applications to improve efficiency, accuracy, and patient care. In an embodiment, the systems and methods are implemented in Al-based triage to assist healthcare providers in quickly and accurately determining the severity of a patient's condition. In another embodiment, the systems and methods are implemented as Al scribe to automate the creation of medical notes during patient visits, and facilitating real-time documentation in various formats such as Subjective, Objective, Assessment and Plan (SOAP) notes. Additionally, the systems and methods provide to automatically prepare patient handouts. The patient handouts include written materials which translate complex medical information into easy-to-understand language to enhance patient comprehension and engagement. To implement these features, the systems and methods may integrate machine learning models and natural language processing engines. The machine learning models are configured to process and analyze real-time patient data and dialogues during consultations, enabling the generation of accurate medical notes and patient handouts. Integration APIs may further facilitate seamless data flow between the AI system and existing healthcare management software, providing that all generated documents are appropriately formatted and stored within the patient's electronic health record system.

In an embodiment, the systems and methods are implemented to generate second opinions to enable healthcare providers to obtain additional insights on diagnoses or treatment plans. Additionally, the systems and methods are implemented in post-visit scenarios. The system is configured to assist in managing follow-up communications by generating draft responses to patient questions. The draft response may then be reviewed and approved by physicians. The system may also generate referral letters, providing that all relevant patient information is accurately conveyed to specialists. In another embodiment, the system is configured to suggest appropriate billing codes based on the documented medical services, for optimizing data processing in financial returns for healthcare providers. The system may execute decision support algorithms and specialized medical knowledge bases to generate reliable second opinions and referral letters. In an embodiment, the system executes automated text generation techniques to create drafts for follow-up communications and referral documents. Additionally, the system may include rule-based engines to analyze documented medical services and accurately suggest appropriate billing codes, streamlining financial processes for healthcare providers. The technical improvements may be utilized individually or collectively within Al-based Electronic Medical Records (EMRs), providing an integrated solution that enhances the capabilities of healthcare providers and optimizes the management of patient care and administrative tasks.

Referring now to FIG. 4E, shown therein is a feature 475 of method 300 as applied within an Al-powered Electronic Medical Record (EMR) Integration Application. In this embodiment, a user, such as a physician, may seamlessly input generated medical information into their EMR system using a GUI element labeled “Send to EMR” button 475. Upon activation of this button, the system initiates a secure transfer process wherein the medical note is copied from the Al-generated output and pasted directly into the appropriate fields within the EMR.

In an embodiment, the method 300 is implemented on a desktop application, referred to as the Integration App. The desktop application establishes a local web server on the user's desktop to facilitate a secure and accurate data transfer. The application incorporates advanced image processing algorithms that interact with the EMR interface to provide precise data entry into the correct fields within the patient records. In an embodiment, once the “Send to EMR” 475 button is clicked, the method includes executing instructions to locate the EMR application window on the user's desktop. The method then brings the Integration App to the foreground for visibility during the integration process. Thereafter, the method includes executing image processing algorithms to identify the exact fields where the Al-generated text needs to be entered. The method provides that the information is accurately embedded into the EMR, thereby improving the reliability and efficiency of the data integration process. The integration provides efficient and accurate incorporation of data into patient records, with improved data synchronization and integrity between the Al-generated outputs and existing EMR systems. Additionally, the EMR application establishes a secure and direct connection between the AI system and desktop-based EMR platforms.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A computer-implemented method of responding to user inputs, the method comprising:
receiving, by a processor, a user input including a category selection and contextual data;
providing, by the processor, an input prompt to a large language model (LLM) based on the user input, the input prompt including a source identifier and one or more instructions;
receiving, by the processor, a LLM output generated in response to the input prompt, the LLM output including data limited to sources identified by the source identifier; and
providing, by the processor, a user output based on the LLM output.

2. The method of claim 1, wherein the source identifier includes a list of trusted websites corresponding to the category selection.

3. The method of claim 1, wherein the LLM output is generated based on natural language processing of the user input.

4. The method of claim 1, wherein the one or more instructions includes an assigned engagement role to the LLM.

5. The method of claim 1, wherein the user input includes a medical inquiry.

6. The method of claim 5, wherein the category selection includes a medical specialty selection.

7. The method of claim 5, wherein the contextual data includes patient triage data.

8. The method of claim 7, wherein the contextual data further includes patient medical history data.

9. The method of claim 1, wherein the user output includes one or more follow-up questions.

10. The method of claim 1, wherein the LLM output includes a first response portion and a second response portion, wherein:
the first response portion is related to the user input and is tailored to a specialized audience; and
the second response portion is a simplified version of the first response portion and is tailored to a general audience.

11. The method of claim 1, wherein the LLM output includes a list of sources used to generate the LLM output.

12. The method of claim 1, wherein the LLM is based on an OpenAI® GPT model.

13. The method of claim 1, wherein the user output is stored in a memory and the method further comprises:
providing, by the processor, a second input prompt to the LLM based on additional contextual data and the stored user output.

14. A system for responding to user inputs, the system comprising:
a processor; and
a non-transitory computer-readable medium having stored thereon instructions which when executed by the processor cause the processor to:
receive a user input including a category selection and contextual data;
provide an input prompt to a large language model (LLM) based on the user input, the input prompt including a source identifier and one or more instructions;
receive a LLM output generated in response to the input prompt, the LLM output including data limited to sources identified by the source identifier; and
provide a user output based on the LLM output.

15. The system of claim 14, wherein the source identifier includes a list of trusted websites corresponding to the category selection.

16. The system of claim 14, wherein the LLM output is generated based on natural language processing of the user input.

17. The system claim 14, wherein the one or more instructions includes an assigned engagement role to the LLM.

18. The system of claim 14, wherein the user input includes a medical inquiry.

19. The system of claim 18, wherein the category selection includes a medical specialty selection.

20. The system of claim 18, wherein the contextual data includes patient triage data.

21. The system of claim 20, wherein the contextual data further includes patient medical history data.

22. The system of claim 14, wherein the user output includes one or more follow-up questions.

23. The system of claim 14, wherein the LLM output includes a first response portion and a second response portion, wherein:
the first response portion is related to the user input and is tailored to a specialized audience; and
the second response portion is a simplified version of the first response portion and is tailored to a general audience.

24. The system of claim 14, wherein the LLM output includes a list of sources used to generate the LLM output.

25. The system of claim 14, wherein the LLM is based on an OpenAI® GPT model.

26. The system of claim 14, wherein the user output is stored in a memory and the instructions, when executed by the processor, further cause the processor to:
provide a second input prompt to the LLM based on additional contextual data and the stored user output.
